(19) United States
(12) Patent Application Publication
(10) Pub. No.: US 2012/0029078 A1
(43) Pub. Date: Feb. 2, 2012

(54) COMPOSITIONS COMPRISING MYRISTIC ACID AND USES THEREOF

(75) Inventors: Robert L. Hesslink, JR., Riverside, CA (US); William P. Spencer, Escondido, CA (US)

(73) Assignee: IMAGENETIX, INC., San Diego, CA (US)

(21) Appl. No.: 13/038,737

(22) Filed: Mar. 2, 2011

Related U.S. Application Data
(60) Provisional application No. 61/309,816, filed on Mar. 2, 2010.

(51) Int. Cl. A61K 31/20; A61P 31/04; A61P 1/02; A61P 29/00

(52) U.S. Cl. 514/558

(57) ABSTRACT

The teachings herein are directed to antibacterial and anti-inflammatory pharmaceutical compositions that include myristic acid. Further embodiments herein are directed to methods of administering sufficient amounts of myristic compositions to patients in need thereof in order to prevent or treat inflammation and/or bacterial infection. Myristic acid has been established to outperform other cetylated fatty acids in treating infection and inflammation, such as 1-TDC which includes cetylated myristic acid. The compositions and methods provided herein can further be used with additional anti-inflammatory, antibacterial, and delivery agents.

### CROSS-REFERENCE TO RELATED APPLICATIONS

The present application claims the benefit of U.S. Provisional Patent Application Ser. No. 61/309,816, entitled “COMPOSITIONS COMPRISING MYRISTIC ACID AND USES THEREOF,” filed Mar. 2, 2010, the entire disclosure of which is hereby incorporated by reference.

### FIELD OF THE INVENTION

The field of the invention is related to antibacterial and anti-inflammatory compositions and methods of administering said compositions to patients in order to prevent or treat inflammation and/or bacterial infection in patients in need thereof.

### BACKGROUND

Periodontitis is a local inflammation that occurs as a result of host responses against specific microorganisms and eventually leads to tissue destruction and systemic complications. Once periodontal inflammation is initiated, the cascade of inflammatory events can proceed in an amplified loop until the infection is contained and injury is confined. In general, the early actions of the host response are later replaced by more specific mechanisms which eventually become redundant in terms of treating the infection. Thus, it is important to limit the host's response and prevent the inflammation from developing into periodontal disease. While it has been shown that many molecules participate in the initiation and development of the host defense mechanisms, the importance of counter-regulatory molecules in the control of inflammatory response has recently been considered.

Fatty acids have been shown to regulate a variety of enzymatic processes that control chronic inflammatory disease. In addition, it has also been shown that fatty acids can decrease the amount of arachidonic acid in cell membranes reducing eicosanoid production via cyclooxygenase and lipoxygenase. The integration between arachidonic acid byproducts and their involvement with leukotriene and prostaglandins leads to inflammation control. These mechanisms have been shown to play important roles in the development of periodontal inflammation. Moreover, high epithelial penetration ability of fatty acids through gingival epithelium suggests that the local application may be favorable in the treatment of periodontal inflammation.

Different cetylated fatty acids play a role in different stages of inflammation. Based on previous in vivo data, it was recognized that the potential suppression of inflammation can be mediated by the topical application of cetylated fatty acids. Recently, 1-tetradecanol complex (1-TDC), a novel monounsaturated fatty acid mixture, which contains a blend of cetylated monounsaturated fatty acids, has been shown to inhibit endothelial activation and reduce tissue responsiveness to cytokines. Preliminary results revealed that 1-TDC significantly inhibits thromboxane A2 production in human recombinant embryonic kidney (HEK)-293 cells through the inhibition of thromboxane synthase receptor, which may suggest inhibiting platelet aggregation through the COX pathway. Accordingly further study is needed to investigate the antibacterial and anti-inflammatory properties of cetylated fatty acids such as 1-TDC and non cetylated fatty acids such as myristic acid (MA).

### SUMMARY OF THE INVENTION

Disclosed are the following methods and compositions. A method of treating an inflammatory condition in a patient in need thereof comprising: providing a therapeutically effective amount of myristic acid in a pharmaceutically acceptable vehicle; administering said myristic acid to said patient, wherein said administering is effective to treat the inflammatory condition.

A method of preventing an inflammatory condition in a patient in need thereof comprising: providing a prophylactic amount of myristic acid in a pharmaceutically acceptable vehicle; administering said myristic acid to said patient, wherein said administering is effective to prevent the inflammatory condition.

A method of treating a bacterial infection in a patient in need thereof comprising: providing a therapeutically effective amount of myristic acid in a pharmaceutically acceptable vehicle; administering said myristic acid to said patient, wherein said administering is effective to treat the bacterial infection.

A composition comprising: a pharmaceutically acceptable vehicle and an effective amount of myristic acid to treat an inflammatory condition in a patient in need thereof.

A composition comprising: a pharmaceutically acceptable vehicle and an effective amount of myristic acid to treat a bacterial infection in a patient in need thereof.

### BRIEF DESCRIPTION OF THE DRAWINGS

It will be appreciated that the drawings are not necessarily to scale, with emphasis instead being placed on illustrating the various aspects and features of embodiments of the invention, in which:

FIG. 1 is a bar graph depicting the anti-bacterial efficacy of myristic acid, compared to other compounds.

FIG. 2 is a line graph establishing myristic acid's antibacterial activity against P. gingivalis is dose-dependent.

FIG. 3 depicts line graphs comparing myristic acid, 1-TDC, 2-TDC, cetyl myristate, and palmityl myristate in their ability to inhibit monocyte-mediated TNF-α, IL-12, IL-1β, IL-8, IL-6, and MCP-1 release over a 6 hour period.

FIG. 4 depicts line graphs comparing myristic acid, 1-TDC, 2-TDC, cetyl myristate, and palmityl myristate in their ability to inhibit monocyte-mediated TNF-α, IL-12, IL-1β, IL-8, IL-6, and MCP-1 release over a 24 hour period.

FIG. 5 depicts line graphs comparing myristic acid, 1-TDC, 2-TDC, cetyl myristate, palmityl oleate, palmityl myristoleate, and palmityl myristate in their ability to inhibit monocyte-mediated TNF-α, IL-12, IL-1β, IL-8, IL-6, and MCP-1 release over a 48 hour period.

FIG. 6 depicts 2 line graphs comparing 1-TDC's ability to inhibit monocyte-mediated cytokine release over 6 and 24 hour periods.

FIG. 7 depicts 2 line graphs comparing myristic acid, 1-TDC, 2-TDC, cetyl myristate, palmityl oleate, palmityl myristoleate, and palmityl myristate in their ability to activate T-lymphocyte-mediated IFN-γ release over 24 and 48 hour periods.

FIG. 8 depicts 2 line graphs comparing myristic acid, 1-TDC, 2-TDC, cetyl myristate, palmityl oleate, palmityl myristoleate, and palmityl myristate in their ability to activate T-lymphocyte-mediated IL-2 release over 24 and 48 hour periods.

FIG. 9 depicts 2 line graphs comparing myristic acid, 1-TDC, 2-TDC, cetyl myristate, palmityl oleate, palmityl myristoleate, and palmityl myristate in their ability to activate T-lymphocyte-mediated IL-10 release over 24 and 48 hour periods.

FIG. 10 depicts 2 line graphs comparing myristic acid, 1-TDC, 2-TDC, cetyl myristate, palmityl oleate, palmityl myristoleate, and palmityl myristate in their ability to activate T-lymphocyte-mediated IL-4 release over 24 and 48 hour periods.

FIG. 11 depicts 2 line graphs comparing myristic acid, 1-TDC, 2-TDC, cetyl myristate, palmityl oleate, palmityl myristoleate, and palmityl myristate in their ability to activate T-lymphocyte-mediated IL-5 release over 24 and 48 hour periods.

### DETAILED DESCRIPTION OF THE ILLUSTRATED EMBODIMENTS

Embodiments of the present invention are described below. It is, however, expressly noted that the present invention is not limited to these embodiments, but rather the intention is that modifications that are apparent to the person skilled in the art and equivalents thereof are also included.

The teachings herein are directed to pharmaceutical compositions of myristic acid and methods of preventing and treating inflammation and/or bacterial infection in a patient in need thereof. Compositions and methods herein can be preferably used to prevent or treat conditions with both infection and inflammation such as periodontitis. As gingivitis (gum inflammation) usually precedes periodontitis (gum disease) the teachings herein can be used to prevent or treat this condition also.

Myristic acid (MA) also known as tetradecanoic acid is a saturated fatty acid with the molecular formula CH₃(CH₂)₁₂COOH. The term “myristic acid” as used herein is expressly not cetylated (esterified with cetyl alcohol) unless designated otherwise as “cetylated myristic acid”. Cetylated myristic acid is a compound found within 1-TDC (available from Imagenetix, Inc, San Diego, Calif.) a proprietary blend of cetylated monounsaturated fatty acids. 1-TDC is disclosed in U.S. Pat. No. 7,612,111 to Spencer et al., and is hereby incorporated by reference in its entirety. 2-TDC is a proprietary blend of monounsaturated fatty acids, similar to 1-TDC except that the fatty acids are not cetylated. As established in the Examples below, myristic acid performed superiorly to 1-TDC and the cetylated fatty acids found in 1-TDC with respect to controlling bacterial infection and in reducing inflammation.

Preferred compositions and methods provided herein include myristic acid as an ingredient in a pharmaceutically acceptable carrier. Myristic acid, to be used herein, can be derived from any suitable source non-exclusively including: nutmeg, palm oil, coconut oil, butter fat, and the like, for example.

While the methods and compositions provided herein are primarily directed to preventing and treating the inflammation and infection associated with periodontitis, as a host's general reaction to bacterial infection is inflammation, any suitable bacterial infection can also be prevented or treated using the teachings herein. According to more specific embodiments, the teachings herein can be used to prevent or treat the infection by bacteria associated with periodontitis such as Porphyromonas gingivalis, Aggregatibacter actinomycetemcomitan, Fusobacterium nucleatum, or any other anaerobic, gram-negative, pathogenic bacterium. Examples of other pathogenic, gram-negative bacteria that the teachings herein can be used against are Bacteroides fragilis, Brucella abortus, Escherichia coli, Haemophilus influenzae, Klebsiella pneumoniae, Neisseria gonorrhoeae, Proteus vulgaris, Pseudomonas aeruginosa, Salmonella typhi, Shigella dysenteriae, and Yersinia pestis. In still other embodiments, the compositions herein can be used to prevent and treat infection by gram-positive bacteria in a patient in need thereof. Methods of preventing a bacterial infection with myristic acid can non-exclusively include the application of ointments on open wounds, administration to a patient prior to a medical procedure, such as surgery, and administration to patient suffering from immune deficiencies.

While preferred embodiments are directed to compositions and methods of treating and preventing both inflammation and infection, the teachings herein can also be used to treat any suitable disorder associated with inflammation in a patient. Such disorders non-exclusively include: acne vulgaris, asthma, autoimmune diseases, chronic inflammation, chronic prostatitis, glomerulonephritis, hypersensitivities, inflammatory bowel diseases, pelvic inflammatory disease, reperfusion injury, rheumatoid arthritis, transplant rejection, and vasculitis. More specifically, preferred compositions and methods are directed to preventing and/or treating inflammatory conditions characterized by overstimulation of one or more of the following proinflammatory cytokines: IL-1β, IL-6, IL-12, IL-8, MCP-1 (monocyte-mediated release) and IL-2 (T-lymphocyte-mediated release) by suppressing one or more of said cytokines. Further preferred compositions and methods are directed to preventing and/or treating inflammatory conditions characterized by suppression of one or more of the following anti-inflammatory cytokines: IL-10, IL-5, and IL-4 (T-lymphocyte-mediated release) by activating one or more of said cytokines.

In general, a patient can first be diagnosed as one that is either susceptible to or suffering from harmful inflammation and/or infection. Patients who are susceptible to inflammation and infection can be determined through examination and/or assessing their risk factors. For example, people susceptible to periodontitis can include one or more of the following non-exclusive risk factors: gingivitis, heredity, poor oral health habits, tobacco use, diabetes, older age, decreased immunity, such as that occurring with leukemia or HIV/AIDS, poor nutrition, certain medications, hormonal changes, such as those related to pregnancy, substance abuse, ill-fitting dental restorations, and lower socioeconomic status.

The patient in need thereof can then be administered a pharmaceutically acceptable composition that includes myristic acid in sufficient amount that either prevents or treats the inflammation and/or infection. Preventing and treating inflammation and/or infection can include one or more of the following: preventing bacterial infection, the killing of infecting bacteria, the suppression of pro-inflammatory pathways, and the activation or stimulation of anti-inflammatory pathways. Myristic acid compositions herein can be packaged to include instructions, dosage, and indication information directing the use of the composition to the treatment or prevention of inflammation and/or infection in a patient in a need.

According to further embodiments, the myristic acid compositions and methods of use provided herein can be used with other anti-inflammatory agents and/or other antibiotics, non-exclusively including: sodium myristate, chlorhexidine, and the like, for example. More specifically myristic compositions can be used with agents that are known for suppressing TNF-α or IFN-γ release, such as 1-TDC, 2-TDC, cetyl myristate, myristic acid, palmityl myristate, palmityl oleate, and palmityl myristoleate.

The compositions of the myristic acid described herein can be formulated as pharmaceutical compositions and administered to a mammalian host, such as a human patient, in a variety of forms adapted to the chosen route of administration, i.e., orally or parenterally, by intravenous, intramuscular, topical or subcutaneous routes.

Such compositions may be systemically administered in vivo by a variety of routes. For example, they may be administered orally, in combination with pharmaceutically acceptable excipients such as an inert diluent or an assimilable edible carrier. They may be enclosed in hard or soft shell gelatin capsules, may be compressed into tablets, or may be incorporated directly with the food of the patient's diet. For oral administration, the active ingredient or ingredients may be combined with one or more excipients and used in the form of ingestible tablets, buccal tablets, troches, capsules, elixirs, suspensions, syrups, wafers, and the like. Pharmaceutical compositions herein can readily include oral care compositions, such as therapeutic mouth rinses, toothpastes, gels, tooth powders, chewing gums, mints, mouth sprays, dissolvable strips, and lozenges comprising at least a minimally effective amount of myristic acid.

Such compositions and preparations should contain at least 0.1% of active compound. The percentage of the compositions and preparations may, of course, be varied and may conveniently be between about 2% to about 60% of the weight of a given unit dosage form. The amount of active ingredient in such useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, a sugar, such as sucrose or fructose, or an artificial sweetener, such as sucralose or aspartame, as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The compositions may also be administered intravenously or intraperitoneally by infusion or injection. Solutions of myristic acid, its salts and other active ingredients can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions can be prepared by incorporation of myristic acid in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions. For topical administration, myristic acid and other active ingredients may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, oils, such as vegetable oil, olive oil and the like, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin, or inside of the mouth of the user.

The myristic acid for topical application can be prepared to be used as a cream or as a solution. In some embodiments, the myristic acid is formed into an emulsion and the emulsion is applied to the treatment site.

Useful dosages of myristic acid can be determined by comparing its in vitro activity and in vivo activity in animal models. Methods for the extrapolation of effective dosages in mice, and other animals, to humans are known to the art; for example. Generally, the concentration of a myristic acid in a liquid composition will be from about 0.1-25 wt-%, preferably from about 0.5-10 wt-%. The concentration in a semi-solid or solid composition such as a cream, a gel, or a powder will be about 0.1-5 wt-%, preferably about 0.5-2.5 wt-%.

The amount of the myristic acid, or an active salt or derivative thereof, required for use alone or with other agents will vary not only with the particular salt selected but also with the route of administration, the nature of the condition being treated and the age and condition of the patient and will be ultimately at the discretion of the attendant physician or clinician.

In general, however, a suitable dose of myristic acid may be in the range of from about 0.5 to about 100 mg/kg, e.g., from about 1 to about 75 mg/kg of body weight per day, or 1.5 to about 50 mg per kilogram body weight of the recipient per day, or about 2 to about 30 mg/kg/day, or about 2.5 to about 15 mg/kg/day.

Myristic acid can be conveniently administered in unit dosage form; for example, containing 5 to 1000 mg, conveniently 10 to 750 mg, most conveniently, 50 to 500 mg of active ingredient per unit dosage form.

Myristic acid can be administered to achieve peak plasma concentrations of the active compound of from about 0.5 to about 75 μM, preferably, about 1 to 50 μM most preferably, about 2 to about 30 μM This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The invention may be embodied in other specific forms besides and beyond those described herein. The foregoing embodiments are therefore to be considered in all respects illustrative rather than limiting, and the scope of the invention is defined and limited only by the appended claims and their equivalents, rather than by the foregoing description.

The following examples are illustrative of the compounds and use of such compounds of the present invention, but it is understood that the invention is not limited thereto.

### Example I

The following experiment establishes the anti-bacterial activity of myristic acid and compares its effects to other agents (e.g., 1-TDC) and controls. P. gingivalis A7436 was cultured on laced blood paromomycin agar plates and then transferred to Schaedler's broth. All cultures were grown at 37° C. under anaerobic conditions. In order to dissolve the 1-TDC and MA in an aqueous medium that can be utilized in carrying out the experiments, the following ingredients were used: ethanol (EtOH) and Methyl-β-cyclodextrin (Sigma-Aldrich, St. Louis, Mo.). More specifically, 750.0 mg of Methyl-β-cyclodextrin was dissolved in 5.0 mL of nano-pure water to get a concentration 150.0 mg/mL (MBC). The MBC solution was stored in the refrigerator at a temperature of 4° C. and was returned to room temperature before use. MA was prepared by dissolving 90.0 mg in 3.0 mL of ETOH, 2.4 mL of MBC, and 24.6 mL of SB in a sterile tube. The entire mixture was placed in a hot bath at 30° C. for 30 minutes. The final concentration of MA was 3.0 mg/mL.

2×10⁷ cells were transferred to 5 mL culture tubes and treated with either 1-TDC or MA (1.0 mg/ml, 0.7 mg/mL, 0.5 mg/mL, 0.3 mg/mL, 0.1 mg/mL) or corresponding vehicle concentrations. A negative control was treated with additional Schaedler's broth. Tubes were incubated in anaerobic jars at 37° C. for several time points between 4-72 hours. The optimal time for growth was found at 24 hours and the subsequent experiments were performed at 24 hours.

After incubation, viability of the P. gingivalis cells was assessed. 1 mL of each sample was removed and spun down. Bacteria were resuspended in 1 mL of 0.85% NaCl solution. To generate a standard curve, half of the bacteria that was not treated with vehicle or compound were heat-killed for one hour and then mixed with live cells in known proportions (0%, 25%, 50%, 75% and 100% alive). The 100% live cells were used as the negative control. To assess the viability, a fluorescent based kit was used. Live/Dead BacLight Bacterial Viability Kit (Invitrogen L7012) allows for differentiation between alive and dead bacteria at a highly accurate and sensitive way. In this assay, SYTO-9 green and propidium iodide (PI) dyes were mixed at a 1:1 ratio and added to all samples (3 μl for 1 mL of cells). The samples were then incubated at room temperature for 15 minutes.

Samples were then run on the flow cytometer (FACScan). Data collection and analysis were performed using BD Cellquest Pro v5.2 software. SYTO-9 stains all cells (green) while PI only stains cells with damaged membranes (red). Thus, in the presence of both PI and SYTO-9, there was a reduction in SYTO-9 staining in dead cells. Using the software, two distinct regions, representing live and dead cells, were clearly visible. The standard curve was used to verify the reliability of the reported percentages. The coefficient of determination for the standard curves was greater than 0.9. From these established regions, percent viability of the experimental samples were determined.

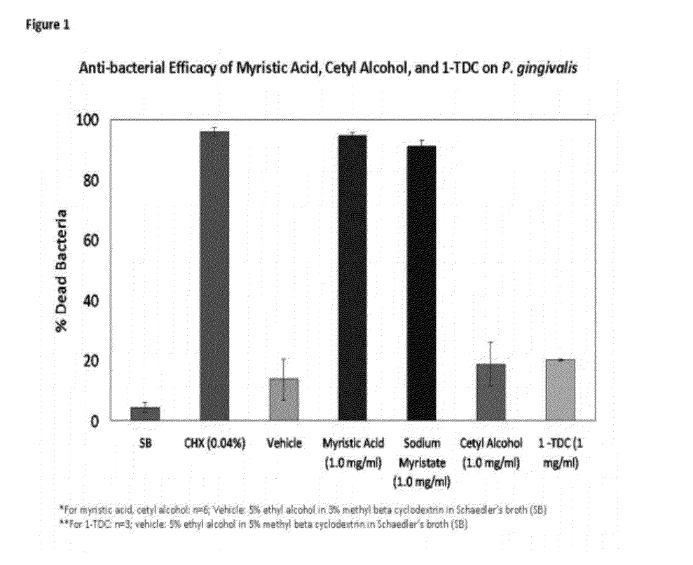

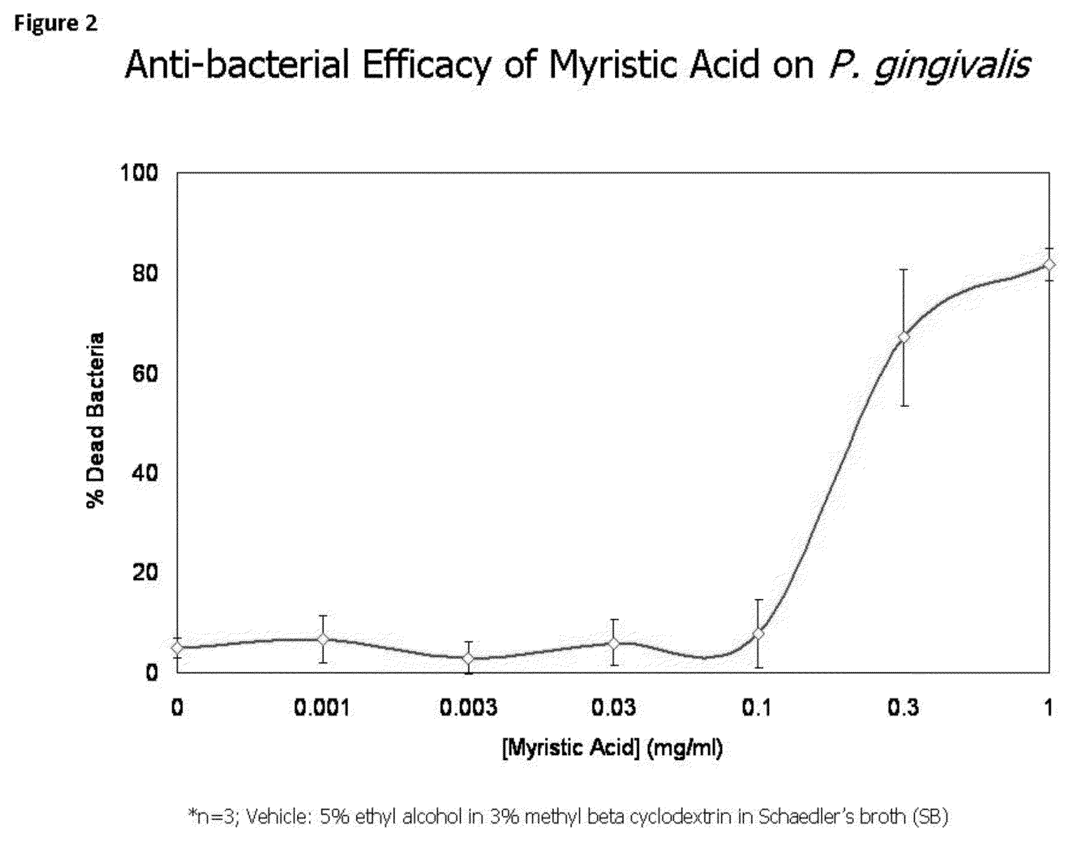

The antibacterial properties of 1-TDC and MA against P. gingivalis were tested and compared to cetyl alcohol, sodium myristate and a positive control chlorhexidine (CHX) (0.04%). FIG. 1 shows percentage of dead bacteria (P. gingivalis) when treated with the above listed compounds. As shown, myristic acid together with sodium myristate showed a comparable level of antibacterial capacity as seen with chlorhexidine. 1-TDC however, did not show more than 20% killing efficacy on P. gingivalis. As shown in FIG. 2, the results indicate that myristic acid's antibacterial activity is dose-dependent and increases dramatically at 0.1 mg/ml dose and higher doses (e.g., 1 mg/ml).

### Example II

Cytokine release from monocytes was measured and assessed in order to determine the anti-inflammatory effects of cetylated fatty acids such as 1-TDC and non-cetylated fatty acids such as myristic acid. Human primary monocytes were provided by healthy subjects (n=7) who were not taking medication, had no medical condition, and no periodontal or gingival inflammation. None of the subjects were smokers and all subjects were of Caucasian origin with an age range of 24-51 years old. All patient samples were obtained after the approval of the Institutional Review Board at Boston University Medical Center.

Fresh peripheral venous blood (−72 ml) was obtained by venipuncture into heparinized (10 U/mL) glass tubes. Monocytes were isolated using Ficoll-Hypaque density gradient centrifugation and separated from the other mononuclear cells (e.g., lymphocytes) by adherence for over 2 hours. Pure cell cultures were treated with various doses (10⁻⁵-10⁻⁹ M) of various types of fatty acids such as 1-TDC and myristic acid for 30 minutes. Vehicle (5% ethyl alcohol), which was used to dissolve the compound into an aqueous preparation, was used as the negative control while dexamethasone (1 nM) was used as the positive control. After incubation with the test compound, half of the samples were treated with LPS from E. coli (100 ng/mL) as the activator of cell cytokine release over various time points (24 hours) at 37° C. under 5% CO₂. Supernatants were collected and stored at −80° C. until analyzed. Each sample was prepared in triplicate. Cytokine release (IL-1β, TNF-α, IL-6, IL-12, IL-8, and MCP-1) was analyzed by xMAP multiplexing technology using Luminex 100 Platform. Data was presented as % inhibition over the vehicle's effect of LPS-mediated cell activation. Dexamethasone inhibition was taken as 100% inhibition.

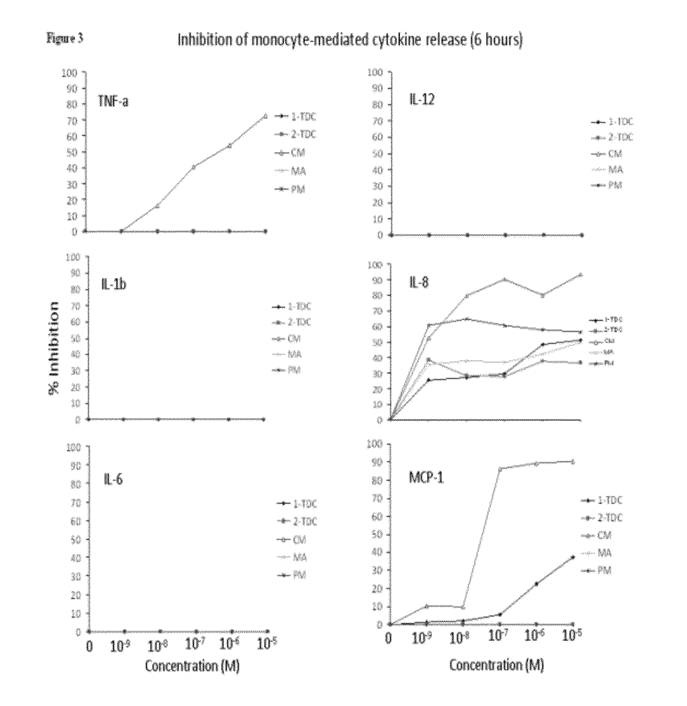

Cytokine release data were gathered at 6, 24, and 48 hour time points for monocyte/macrophages (FIGS. 3-5). Each experiment was repeated at least 3 times and data was presented as percentage of inhibition over the vehicle's effect of LPS-mediated cell activation. At least 10% inhibition is considered a significant inhibitory effect to demonstrate the potential impact of these compounds.

The inhibition potential of various compounds (i.e., 1-TDC, 2-TDC, cetyl myristate (CM), myristic acid (MA), palmityl myristate (PM), palmityl oleate (PO), and palmityl myristoleate (PMO)) on monocyte-mediated cytokine release was tested at different concentrations and is shown in Table 1 and Table 2 below. Table 1 shows that cetyl myristate inhibits TNF-α as early as 6 hours. IL-8, which is a strong chemoattractant and mainly released by neutrophils, was also detected at high levels at 6 hours and was significantly inhibited by all compounds tested. MCP-1, which is also a strong chemokine for monocytes, was inhibited by 1-TDC and cetyl myristate. None of the other tested compounds were strong inhibitors of the monocyte-mediated cytokine release at 6 hours.

TABLE 1
Inhibition of cytokine release from peripheral blood monocytes over a 6
hour period.

| | | | | | | |
|---|---|---|---|---|---|---|
| Compound | TNF-α | IL-1β | IL-6 | IL-12 | IL-8 | MCP-1 |

| | | | |
|---|---|---|---|
| 1-TDC | | ✓ | ✓ |
| 2-TDC | | ✓ | |
| Cetyl Myristate | ✓* | ✓ | ✓ |
| Myristic Acid | | ✓ | |
| Palmityl Myristoleate | | ✓ | |

*Strong inhibition

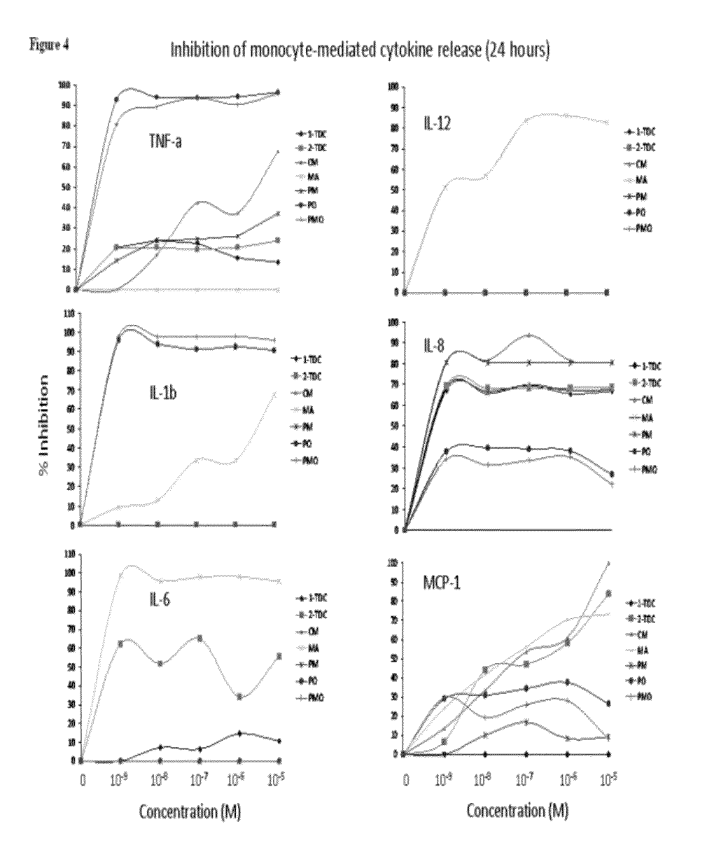

Table 2 below demonstrates the 24-hour inhibitory potential of the tested compounds. At this time point, palmityl oleate and palmityl myristoleate significantly and fully inhibited both TNF-α and IL-1β release. Cetyl myristate's inhibition on TNF-α continued at the 24-hour mark. 1-TDC, 2-TDC, and palmityl myristate also blocked TNF-α release, however the inhibition was weaker compared to cetyl myristate. In addition to TNF-α, cetyl myristate also inhibited IL-8 and MCP-1 release. While all tested compounds inhibited IL-8 release, 2-TDC blocked IL-6 as well, which is a potent pro-inflammatory cytokine released by monocytes and MCP-1. Myristic acid significantly blocked all cytokines except TNF-α at the 24-hour mark. With the sole exception of TNF-α, myristic acid significantly and dose dependently inhibited all other cytokines as shown in FIG. 4.

TABLE 2
Inhibition of cytokine release from peripheral blood monocytes over 24
hours.

| Compound | TNF-α | IL-1β | IL-6 | IL-12 | IL-8 | MCP-1 |
|---|---|---|---|---|---|---|
| 1-TDC | ✓ | | | | ✓ | |
| 2-TDC | ✓ | | ✓ | | ✓ | ✓ |
| Cetyl Myristate | ✓ | | | | ✓ | ✓ |
| Myristic Acid | | ✓ | ✓ | ✓ | ✓ | ✓ |
| Palmityl Myristate | ✓ | | | | ✓ | |
| Palmityl Oleate | ✓ | ✓ | | | ✓ | ✓ |
| Palmityl Myristoleate | ✓ | ✓ | | | ✓ | ✓ |

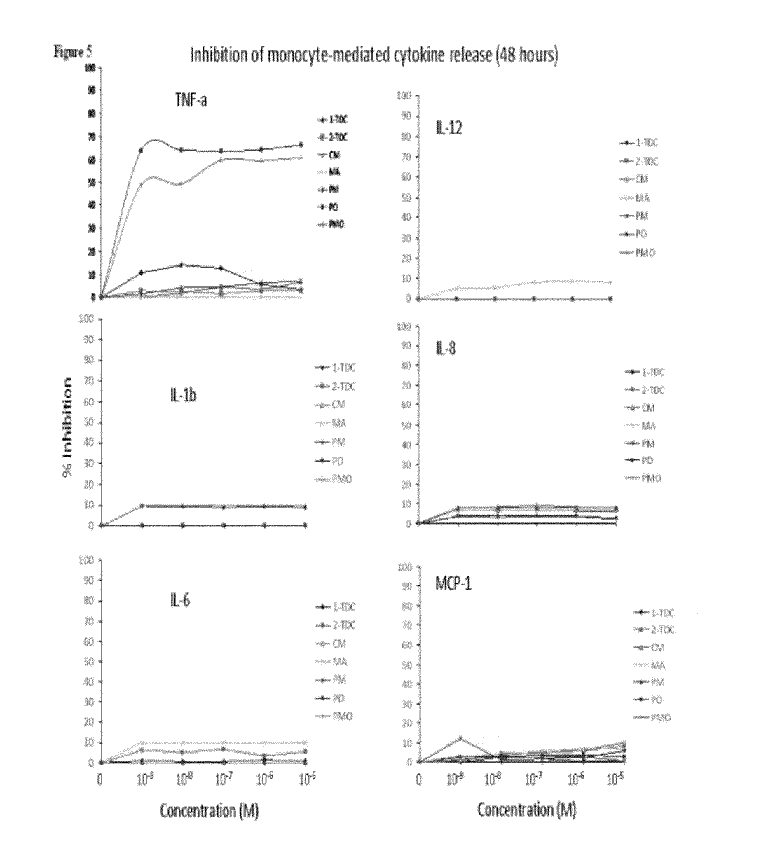

In FIGS. 3-5 dose-response and comparative analyses of the tested compounds can be observed. FIG. 3 demonstrates the 6-hour-response for all mediators detected while FIGS. 4 and 5 show the 24- and 48-hour inhibition profiles of all compounds and their effective concentrations on the same inflammatory mediators, respectively. The 48-hour results demonstrated that the inhibitory potentials of all the compounds detected at 24-hour continued in the same manner but the effect was declined or diminished at the end of the observation period.

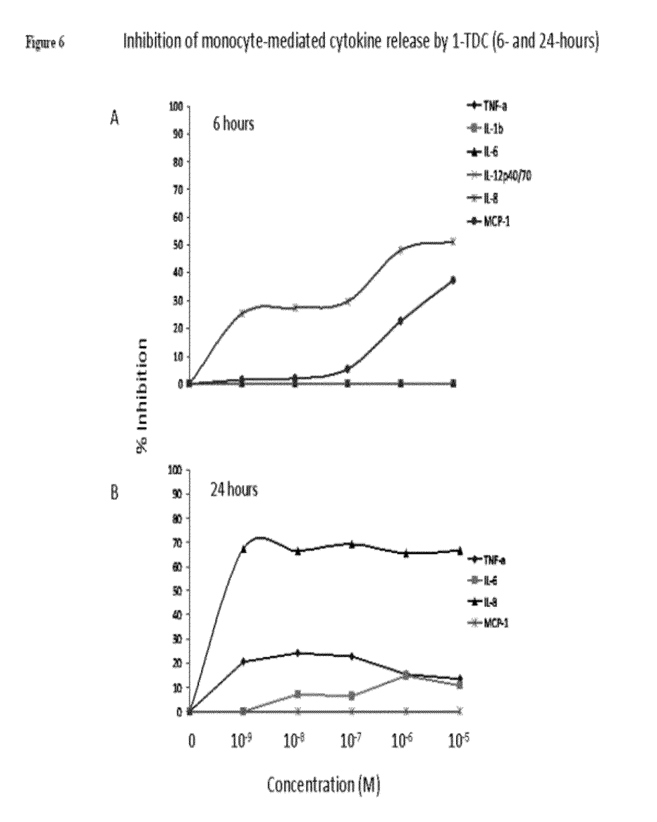

1-TDC's inhibitory effects on monocyte-mediated cytokine release at 6-hours and 24-hours is shown in FIG. 6, Panels A and B respectively. Results demonstrated that 1-TDC significantly inhibited the release of TNF-α, at 24-hours and IL-8 at both of the 6- and 24-hour periods. While the inhibitory effect of 1-TDC on IL-8 at 6-hour was dose-dependent, there was no significant difference between various doses of 1-TDC at 24 hours for IL-8 and TNF-α inhibitions. MCP-1 was inhibited significantly only at 6 hours by 1-TDC. No inhibition above 20% was detected for IL-1β, IL-6, and IL-12 (data not shown). These results bolstered previous in vivo results where the inflammatory changes induced by P. gingivalis were reduced by topical 1-TDC treatment.

### Example III

Cytokine release from T-lymphocytes was measured and assessed in order to determine the anti-inflammatory effects of cetylated fatty acids such as 1-TDC and uncetylated fatty acids such as myristic acid. More specifically, the following compounds were tested: 1-TDC, 2-TDC, cetyl myristate (CM), myristic acid (MA), palmityl myristate (PM), palmityl oleate (PO), and palmityl myristoleate (PMO). For this experiment, peripheral blood mononuclear cells were isolated from healthy donors (n=8) with no known medication use by Ficoll-Hypaque density gradient centrifugation. Primary T-lymphocytes were separated from other mononuclear cells (e.g., monocytes) by negative selection using magnetic cell sorting (Dynal, Invitrogen) and pure cell cultures were treated with various doses of tested compounds for 30 minutes. The tested doses were 10⁻⁵-10⁻⁹M. Vehicle, which was used to dissolve the compounds into an aqueous preparation, was used as the negative control while dexamethasone (1 nM) was used as the positive control. After the incubation with test compounds, half the samples were treated with DynaBeads (Dynal, Invitrogen) coated with CD3 and CD28 antibodies as the activator of cell cytokine release. Cells were incubated over various time points at 37° C. under 5% CO₂. Supernatants were collected at 24 and 48 hours and were stored at −80° C. until analyzed. Each sample was prepared in triplicates. The cytokine release was analyzed by xMAP multiplexing technology using Luminex 100 platform. This method allowed simultaneous analyses of all the cytokines proposed in this experiment.

The following T-lymphocyte associated cytokines: IFN-γ, IL-2, IL-10, IL-5, and IL-4 were assessed. Out of these molecules, IFN-γ and IL-2 are considered to be the traditional cytokines released from T-helper 1 (Tₕ1) cells and represent a “pro-inflammatory” activation while IL-10, IL-4, and IL-5 are released from T-helper 2 (Tₕ2) cells and considered to be “anti-inflammatory”. A paradigm shift between the cytokines by Tₕ1 and Tₕ2 indicates a more or less inflammatory process depending on which of these cytokines are increased. While the validity of this shift is questioned in different diseases and/or infections, the central role of T cells in the progression of inflammation is still evaluated on the cytokine release by these subsets of lymphocytes.

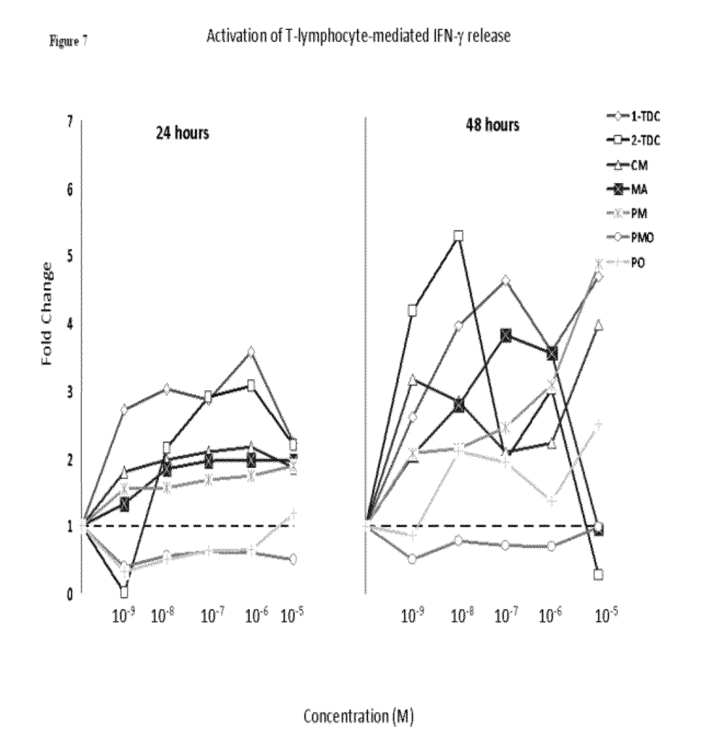

Data was gathered throughout 24- and 48-hour time points for T-lymphocyte mediated release. The lymphocyte responses for all tested compounds are shown in FIGS. 7-11. Each experiment was repeated at least 3 times and data was presented as %-inhibition over the vehicle's effect of LPS-mediated cell activation. At least 10% inhibition is considered as inhibitory effect to demonstrate the potential impact of these compounds.

FIG. 7 shows that with the exception of the palmityl myristoleate, each of the tested compounds have the capacity to activate IFN-γ release by T-lymphocytes and this effect increases over time. Palmityl myristoleate (both at the 24 hour mark and the 48 hour mark) and Palmityl oleate (at the 24 hour mark) inhibit IFN-γ production by T-lymphocytes. 2-TDC had the highest potency of activation of IFN-γ in 48 hours, although this effect does not represent a dose-dependent change. On the other hand, 1-TDC, cetyl myristate, and palmityl myristate exerted a dose-dependent activation on T-cell IFN-γ release.

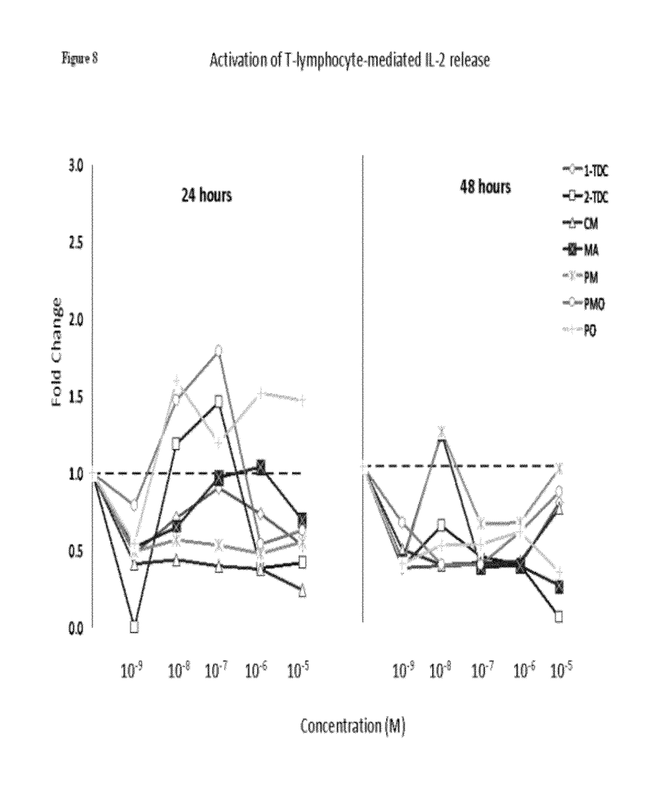

FIG. 8 depicts the effects of the tested compounds on T-lymphocyte IL-2 production. Compared to the IFN-γ results, the tested compounds effect on IL-2 production was considerably less when cells were treated with various doses of different monounsaturated fatty acids. Interestingly, the tested compounds that activated IFN-γ release inhibited IL-2 production, while IFN-γ inhibitors activated IL-2 release over a 24 hour period, including myristic acid. Over 48 hours, all of the tested compounds suppressed the generation of IL-2 by human T lymphocytes.

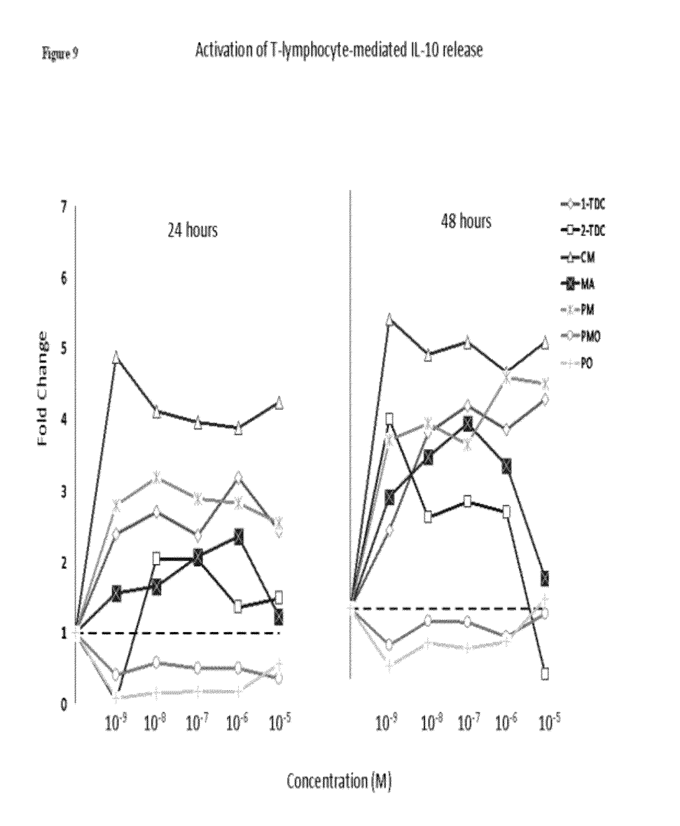

FIG. 9 shows the tested compounds effect on IL-10 generation by T lymphocytes. With the exception of palmityl myristoleate and palmityl oleate, all the tested compounds generated significant T-lymphocyte release of the “anti-inflammatory” cytokine IL-10. This activation effect is potent since even the lowest doses of the tested compounds (10⁻⁹M) increased IL-10 production, and the increase was stable over time. Cetyl myristate was shown to be the most potent activator of IL-10 production compared to the other tested compounds.

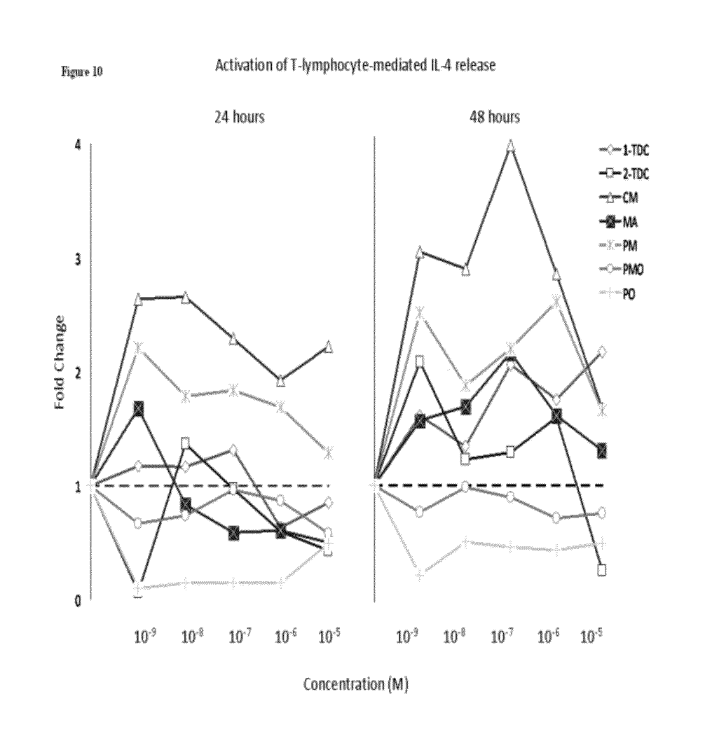

FIG. 10 demonstrates the effects of the tested compounds on another well-known anti-inflammatory cytokine released by the human T cells: IL-4. The results for IL-4 were similar to those seen with IL-10 as most of the tested compounds were potent activators of IL-4 generation. However it was noted that the fold-change over baseline and vehicle normalization was less than the IL-10 results. Cetyl myristate was shown to be the most potent compound tested with regards to IL-4 generation.

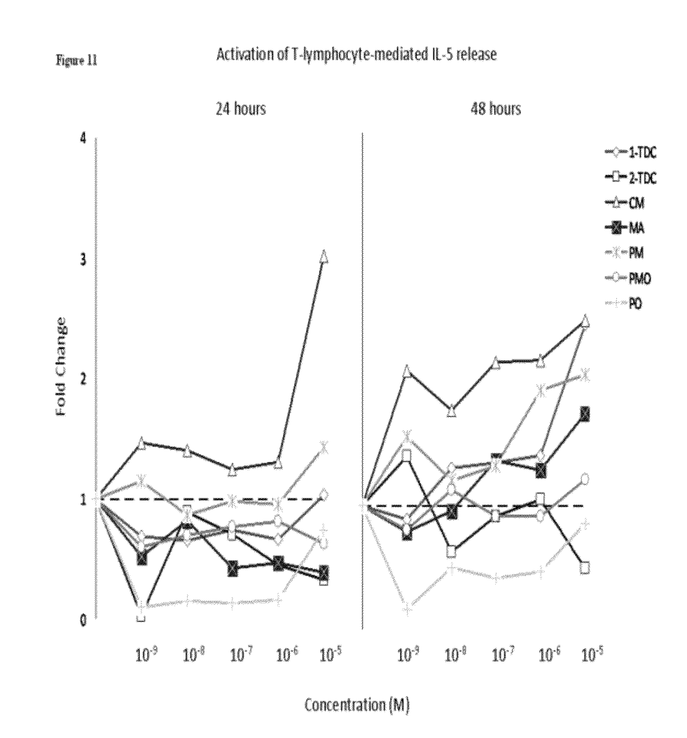

FIG. 11 demonstrates the effects of the tested compounds on another well-known anti-inflammatory cytokine released by the human T cells: IL-5. The IL-5 results were consistent with the results shown in FIGS. 9 and 10 with respect to Tₕ2-mediated generation of anti-inflammatory cytokines as the major target for the cetylated monounsaturated fatty acids in controlling inflammation. Again, cetyl myristate was shown to be the most potent activator of IL-5 release. Myristic acid, was shown to have the capacity to activate IFN-γ release by T cells and this effect increased over time.

These working examples show that myristic acid, can significantly act as both an antibacterial and anti-inflammatory agent by both suppressing anti-inflammatory cytokines and activating pro-inflammatory cytokines. Myristic acid thus has utility in the prevention and treatment of inflammatory conditions initiated by bacteria, especially by P. gingivalis.

### Example IV

Fifteen New-Zealand White rabbits are distributed into 3 groups: (1)

No treatment (5 animals), (2) Placebo treatment (5 animals), and (3) myristic acid treatment (5 animals). In all animals, periodontal disease is established by P. gingivalis application every-other-day over a 6-week period. At 6 weeks, P. gingivalis application is terminated and topical use of the myristic acid and placebo agents is initiated for a second 6 weeks at which time animals are sacrificed. Morphometric, radiographic, and histologic evaluations are performed. Histologic sections are stained with hematoxylin-eosin, and tartrate-resistant acid phosphatase (TRAP) for descriptive histology and osteoclast activity. In addition, osteocalcin staining is used to detect osteoblast activity.

Topical delivery of myristic acid preparation stops the progression of gingival inflammation and bone destruction induced by P. gingivalis. Myristic acid treated animals also reform soft and bone tissues lost to periodontal inflammation. These results are compared with histomorphometric assessments where treatment with myristic acid results in significant changes in tissue and bone level compared to placebo and no treatment groups. Hematoxylin-eosin stained sections indicate a complete reversal of inflammatory changes, whereas the placebo and no treatment groups demonstrate a progression of periodontal inflammation. Myristic acid significantly suppresses the osteoclastic activity and results in increased osteoblastic activity suggesting new bone formation.

What is claimed is:

1. A method of treating an inflammatory condition in a patient in need thereof comprising: providing a therapeutically effective amount of myristic acid in a pharmaceutically acceptable vehicle; administering said myristic acid to said patient, wherein said administering is effective to treat the inflammatory condition.

2. The method of claim 1, wherein the patient in need thereof is, prior to administration, identified as suffering from an inflammatory condition.

3. The method of claim 1, wherein the patient in need thereof is also suffering from, or is susceptible to, a bacterial infection.

4. The method of claim 3, wherein administering said myristic acid is effective to treat or prevent the bacterial infection.

5. The method of claim 4, wherein the bacterial infection is caused by one or more gram-negative bacteria.

6. The method of claim 4, wherein the bacterial infection is caused by bacteria selected from the group consisting of: Porphyromonas gingivalis, Actinobacillus actinomycetemcomitans, and Fusobacterium nucleatum.

7. The method of claim 1, wherein the inflammatory condition is periodontitis.

8. The method of claim 1, wherein the myristic acid is effective in
1) suppressing the release of one or more proinflammatory cytokines;
2) suppressing the T-lymphocyte-mediated release of the proinflammatory cytokine IL-2; or
3) suppressing the monocyte-mediated release of one or more of the proinflammatory cytokines selected from the group consisting of: IL-113, IL-6, IL-12, IL-8, and MCP-1.

9. The method of claim 1, wherein the myristic acid is effective in activating the release of one or more anti-inflammatory cytokines in the patient in need thereof.

10. The method of claim 9, wherein the myristic acid is effective in activating the T-lymphocyte-mediated release of one or more anti-inflammatory cytokines selected from the group consisting of: IL-10, IL-5, and IL-4.

11. A method of preventing an inflammatory condition in a patient in need thereof comprising: providing a prophylactic amount of myristic acid in a pharmaceutically acceptable vehicle; administering said myristic acid to said patient, wherein said administering is effective to prevent the inflammatory condition.

12. The method of claim 11, wherein the inflammatory condition is periodontitis.

13. The method of claim 11, wherein the myristic acid is effective in activating the T-lymphocyte-mediated release of one or more anti-inflammatory cytokines selected from the group consisting of: IL-10, IL-5, and IL-4.

14. A composition comprising: a pharmaceutically acceptable vehicle and an effective amount of myristic acid to treat an inflammatory condition or a bacterial infection in a patient in need thereof.
